(19) United States
(12) Patent Application Publication
(10) Pub. No.: US 2003/0219831 A1
(43) Pub. Date: Nov. 27, 2003

(54) Split-hybrid system

(76) Inventor: Hsiu-Ming Shih, San-Chung (TW)

Correspondence Address: FISH & RICHARDSON PC, 225 FRANKLIN ST, BOSTON, MA, 02110, US

(21) Appl. No.: 10/445,169

(22) Filed: May 23, 2003

Related U.S. Application Data
(60) Provisional application No. 60/382,984, filed on May 24, 2002.

(51) Int. Cl. G01N 33/53; C07H 21/04; C12N 5/00

(52) U.S. Cl. 435/7.1; 435/69.1; 435/320.1; 435/325; 536/23.2

(57) ABSTRACT

A split-hybrid system that contains a nucleic acid including a promoter, a transcriptional regulatory sequence operably linked to the promoter and having a protein-binding domain, and a reporter gene operably linked to the promoter; a first recombinant polypeptide including a DNA-binding domain that binds to the protein-binding domain and a bait polypeptide; and a second recombinant polypeptide including a transcriptional repression domain and a prey polypeptide that interacts with the bait polypeptide. Interaction between the bait and prey polypeptides represses the expression of the reporter gene, and disruption of the interaction between the bait and prey polypeptides activates the expression of the reporter gene.

### RELATED APPLICATION INFORMATION

This application claims priority to U.S. provisional application serial No. 60/382,984, filed May 24, 2002.

### BACKGROUND

Protein-protein interactions are an intrinsic part of nearly every cellular process. These interactions occur both extracellularly (e.g., ligand-receptor binding, cell adhesion, antigen recognition, and virus-host recognition) and intracellularly (e.g., formation of multiple-protein complexes involved in signal transduction, transcription, translation, and DNA repair and replication). Abnormal protein-protein interactions are often involved in disease development. Such protein-protein interactions are thus potential targets for therapeutic intervention.

### SUMMARY

The present invention relates to a split-hybrid system that can be used for identifying compounds disrupting protein-protein interactions.

In one aspect, this invention features a split-hybrid system that contains (1) a nucleic acid including a promoter, a transcriptional regulatory sequence operably linked to the promoter and having a protein-binding domain, and a reporter gene operably linked to the promoter; (2) a first recombinant polypeptide including a DNA-binding domain that binds to the protein-binding domain and a bait polypeptide; and (3) a second recombinant polypeptide including a transcriptional repression domain and a prey polypeptide that interacts with the bait polypeptide. Interaction between the bait and prey polypeptides represses the expression of the reporter gene, and disruption of the interaction between the bait and prey polypeptides activates the expression of the reporter gene.

A “promoter” is a nucleic acid sequence capable of initiating transcription. It can be a cellular promoter (e.g., the thymidine kinase (TK) promoter) or a viral promoter (e.g., the cytomegalovirus (CMV) promoter and Rous sarcoma virus (RSV) promoter).

A transcriptional regulatory sequence “operably linked to” a promoter is a nucleic acid sequence capable of regulating (i.e., enhancing or inhibiting) the activity of the promoter. A “protein-binding domain” is a nucleic acid sequence that a protein can bind, e.g., the Gal4-binding site.

A “reporter gene” is a gene the expression of which can be conveniently determined, e.g., the luciferase gene (Luc), secreted alkaline phosphatase gene (SEAP), and red elongated 1 gene (RED1). A reporter gene “operably linked to” a promoter is under transcriptional control of the promoter.

A “DNA-binding domain” is an amino acid sequence capable of binding a DNA sequence, e.g., the N-terminal region (amino acids 1-147) of the Gal4 protein (Gal4N).

A “transcriptional repression domain” is an amino acid sequence capable of suppressing the transcriptional activity of a promoter, e.g., the Max binding protein (Mad) and Silencing mediator of retinoic acid and thyroid hormone receptor (SMRT).

A “bait” polypeptide and a “prey” polypeptide are a pair of polypeptides that interact with each other, e.g., the retinoid acid receptor (RAR) and its interacting domain II (IDII), the transforming growth factor-β type I receptor (TRI) and FK506 binding protein (FKBP12), and β-catenin and the T cell factor (TCF). The interaction between a bait polypeptide and a prey peptide can be inhibited by a compound (e.g., a nucleic acid, a polypeptide, a small peptide, a ligand, an antibody, a small molecule, a lipid, a carbohydrate or a natural product extract isolated from an animal, a plant, a fungus, or a microbe). The inhibition can be achieved by binding of the compound to the bait or prey polypeptide.

In another aspect, this invention features a cell (e.g., a mammalian cell) that contains (1) a first nucleic acid including a promoter, a transcriptional regulatory sequence operably linked to the promoter and having a protein-binding domain, and a reporter gene operably linked to the promoter; (2) a second nucleic acid encoding a first recombinant polypeptide including a DNA-binding domain that binds to the protein-binding domain and a bait polypeptide; and (3) a third nucleic acid encoding a second recombinant polypeptide including a transcriptional repression domain and a prey polypeptide that interacts with the bait polypeptide. Interaction between the bait and prey polypeptides represses the expression of the reporter gene, and disruption of the interaction between the bait and prey polypeptides activates the expression of the reporter gene.

Also within the scope of this invention is a method of identifying a compound that disrupts interaction between a bait polypeptide and a prey polypeptide. The method includes providing a split-hybrid system or a cell described above and contacting the system or the cell with a compound. The expression level of the reporter gene, if higher in the presence of the compound than in the absence of the compound, indicates that the compound disrupts the interaction between the bait and prey polypeptides.

The present invention provides a system and method for identifying compounds disrupting protein-protein interactions, e.g., via high throughput screening. The compounds thus identified can be used for treating diseases involving abnormal protein-protein interactions. For instance, an effective amount of a nucleic acid encoding a therapeutic protein can be administered to a subject in need thereof in a gene therapy treatment.

The details of one or more embodiments of the invention are set forth in the accompanying description below. Other advantages, features, and objects of the invention will be apparent from the detailed description, and from the claims.

### DETAILED DESCRIPTION

The present invention provides a method for identifying compounds that disrupt protein-protein interactions using a split-hybrid system. The system contains three elements: a nucleic acid including a promoter, a transcriptional regulatory sequence operably linked to the promoter and having a protein-binding domain, and a reporter gene operably linked to the promoter; a first recombinant polypeptide including a DNA-binding domain that binds to the protein-binding domain and a bait polypeptide; and a second recombinant polypeptide including a transcriptional repression domain and a prey polypeptide that interacts with the bait polypeptide. Interaction between the bait and prey polypeptides represses the expression of the reporter gene, and disruption of the interaction between the bait and prey polypeptides activates the expression of the reporter gene. The elements of the system and the method are described in detail below:

Recombinant Polypeptide Including a DNA-Binding Domain and a Bait Polypeptide

Any polypeptide that binds a defined DNA sequence can be used as a DNA-binding domain. The DNA-binding domain can be derived from a naturally occurring DNA-binding protein, e.g., a prokaryotic or eukaryotic DNA-binding protein. Alternatively, the DNA-binding domain can be a polypeptide derived from a protein artificially engineered to interact with specific DNA sequences.

Examples of DNA-binding domains from naturally occurring eukaryotic DNA-binding proteins include p53, Jun, Fos, GCN4, and Gal4. The DNA-binding domain can also be generated from viral proteins, such as the pappillomavirus E2 protein. In addition, the DNA-binding domain can be derived from a prokaryote, e.g., the E. coli LexA repressor can be used, or the DNA-binding domain can be from a bacteriophage, e.g., a lambda cI protein. Exemplary prokaryotic DNA-binding domains include DNA-binding portions of the P22 Arc repressor, MetJ, CENP-B, Rap1, Xy1S/Ada/AraC, Bir5 and DtxR.

The DNA-binding protein also can be a non-naturally occurring DNA-binding domain generated using recombinant techniques. Methods of generating novel DNA-binding proteins that can selectively bind to a specific DNA sequence are known in the art. See, e.g., U.S. Pat. No. 5,198,346.

The basic requirements of the recombinant protein include the ability to specifically bind a defined nucleotide sequence (i.e., a DNA-binding site) upstream of an appropriate reporter gene. It should cause little or no alteration of the transcriptional activity of the reporter gene by itself. It is also desirable that the bait polypeptide not interfere with the ability of the DNA-binding domain to bind to its binding site on DNA.

When appropriate, the DNA-binding domain can include oligomerization motifs. It is known in the art that certain transcriptional regulators dimerize. Dimerization promotes cooperative binding of the transcriptional regulators to their cognate binding sites on DNA. For example, when a LexA DNA-binding domain is used, it can include a LexA dimerization domain. This dimerization domain facilitates efficient LexA dimer formation and optimizes the efficiency of binding (Golemis and Brent (1992) Mol. Cell Biol. 12:3006). Other exemplary oligomerization motifs include the tetramerization domain of p53 and the tetramerization domain of BCR-ABL.

The bait polypeptide can be any protein of interest and include proteins of unknown, known, or suspected diagnostic, therapeutic, or pharmacological importance. For example, the protein of interest can be a protein suspected of being an inhibitor or an activator of a cellular process (e.g., receptor signaling, apoptosis, cell proliferation, cell differentiation, or import or export of toxins and nutrients). Examples of bait polypeptides can include oncoproteins such as myc, Ras, Src, Fos; tumor-suppressor proteins such as p53, p21, p16, Rb, and constitutively active Rb with deleted phosphorylation sites (Knudsen et al. (1999) Oncogene 18:5239-45); proteins involved in cell-cycle regulation such as kinases (e.g., TRI) and phosphatases; or proteins involved in signal transduction, e.g., IDII, β-catenin, Zap-70, or SAM-68. The full length of the protein of interest, or a portion thereof, can be used as a bait polypeptide. In the instance when the protein of interest is of a large size, e.g., has a molecular weight of over 20 kDa, it can be more convenient to use a portion of the polypeptide.

An unstructured polypeptide linker region can be present between the DNA-binding domain and the bait polypeptide sequence. The linker can facilitate the DNA-binding domain to interact with its binding site on DNA and the bait polypeptide to interact with a prey polypeptide.

The recombinant polypeptide can be produced from a vector containing a nucleic acid sequence that encodes a DNA-binding domain fused in-frame to a bait polypeptide. For production in a mammalian cell, suitable expression vectors are known in the art, e.g., pSG424 (Sadowski and Ptashne (1989) Nucleic Acid Research, 17:7539) and pM (Clontech, Palo Alto, Calif.). The expression vector can include one or more regulatory sequences (e.g., enhancers) operably linked to a promoter that directs the expression of the recombinant polypeptide. The vector can also include a selectable marker, the expression of which in the cell permits selection of cells containing the marker gene from cells that do not contain the marker gene. Selectable markers are known in the art, e.g., neomycin, zeocin, and blasticidin. The vector can be either present as an extrachromosomal DNA or integrated into a chromosome of the cell. Recombinant polypeptide including a transcriptional repression domain and a prey polypeptide

Any of a number of transcriptional repression domains can be used in the recombinant polypeptide. The transcriptional repression domain can be a naturally occurring transcriptional repression domain, e.g., a transcriptional repression domain that is derived from a eukaryotic or prokaryotic source. Exemplary transcriptional repression domains include SMRT and Mad.

A prey polypeptide interacts with a bait polypeptide. This protein-protein interaction links the DNA-binding domain fused to the bait polypeptide with the transcriptional repression domain fused to the prey polypeptide, generating a protein complex capable of directly suppressing the expression of a reporter gene.

The recombinant polypeptide can be produced from a vector containing a nucleic acid sequence that encodes a transcriptional repression domain fused in-frame to a prey polypeptide as described above. The vector can also include a nuclear localization sequence (e.g., those derived from Gal4 and MATα2 genes). The nuclear localization sequence optimizes the efficiency with which the recombinant polypeptide reaches the nuclear-localized reporter gene construct.

Reporter Gene

Expression of a reporter gene indicates interaction between the bait and prey polypeptides. Any suitable reporter gene can be used in a split-hybrid system. Examples include chloramphenicol acetyl transferase (CAT; Alton and Vapnek (1979) Nature 282:864-869), and other enzyme detection systems, such as β-galactosidase; firefly luciferase (deWet et al. (1987) Mol. Cell. Biol. 7:725-737); bacterial luciferase (Engebrecht and Silverman (1984) PNAS 1:4154-4158; Baldwin et al. (1984) Biochemistry 23:3663-3667); phycobiliproteins (especially phycoerythrin); alkaline phosphatases (Toh et al. (1989) Eur. J Biochem. 182:231-238; Hall et al. (1983) J. Mol. Appl. Gen. 2:101), secreted alkaline phosphate (Cullen and Malim (1992) Methods in Enzymol. 216:362-368) or fluorescent proteins (e.g., GFP). Other examples of suitable reporter genes include those encoding surface antigens (e.g., CD16) and drug resistance genes (e.g., HPRT).

The amount of transcription from the reporter gene can be measured using any suitable method known in the art. For example, specific mRNA expression can be detected using Northern blots, and specific protein product can be identified using an antibody. The protein encoded by the reporter gene can also be detected by analysis of an intrinsic activity associated with that protein. For instance, the reporter gene can encode a gene product that, by enzymatic activity, gives rise to a detection signal based on fluorescence, color, or luminescence.

It can be desirable to provide two or more reporter gene constructs which are regulated by interaction of the bait and prey polypeptides, e.g., GFP and BFP reporter genes. The simultaneous expression of the various reporter genes provides a means for distinguishing actual interaction of the bait and prey polypeptides from, e.g., mutations or other spurious events that activate the reporter gene.

The reporter gene can be carried on a suitable vector. This vector can be maintained in a cell (e.g., a mammalian cell) episomally or integrated into a chromosome of the cell.

Host Cells

The split-hybrid system can be used both in vitro and in vivo. Any cultured cells (e.g., primary, secondary, or immortalized mammalian cells) can be employed for the in vivo use of the split-hybrid system. Exemplary mammalian cells are those of mouse, hamster, rat, rabbit, dog, cow, and primate including human. They can be of a wide variety of tissue types, including mast cells, endothelial cells, hepatic cells, kidney cells, and other cell types.

“Primary cells” are cells isolated from a mammal (e.g., from a tissue source) and grown in culture for the first time before subdivision and transfer to a subculture. “Secondary cells” are cells at all subsequent steps in culturing. Examples of mammalian primary and secondary cells that can be transfected include fibroblasts, keratinocytes, epithelial cells (e.g., mammary epithelial cells and intestinal epithelial cells), endothelial cells, glial cells, neural cells, formed elements of the blood (e.g., lymphocytes and bone marrow cells), muscle cells and precursors of these somatic cell types.

“Immortalized cells” are cell lines that exhibit an apparently unlimited lifespan in culture. Examples of immortalized human cell lines useful for the present mammlian split-hybrid system include, but are not limited to, CV-1 cells, HT1080 cells (ATCC CCL 121), HeLa cells and derivatives of HeLa cells (ATCC CCL 2, 2.1 and 2.2), MCF-7 breast cancer cells (ATCC BTH 22), K-562 leukemia cells (ATCC CCL 243), KB carcinoma cells (ATCC CCL 17), 2780AD ovarian carcinoma cells (Van der Blick et al. (1988) Cancer Res 48:5927-5932), Raji cells (ATCC CCL 86), Jurkat cells (ATCC TIB 152), Namalwa cells (ATCC CRL 1432), HL-60 cells (ATCC CCL 240), Daudi cells (ATCC CCL 213), RPMI 8226 cells (ATCC CCL 155), U-937 cells (ATCC CRL 1593), Bowes Melanoma cells (ATCC CRL 9607), WI-38VA13 subline 2R4 cells (ATCC CLL 75.1), and MOLT-4 cells (ATCC CRL 1582), as well as heterohybridoma cells produced by fusion of human cells and cells of another species. Secondary human fibroblast strains, such as WI-38 (ATCC CCL 75) and MRC-5 (ATCC CCL 171) can also be used.

Methods of transfecting vectors described above into a cell can be carried out using procedures known in the art. Examples of mammalian cell transfection methods include calcium phosphate and calcium chloride co-precipitation, DEAE-dextran-mediated transfection, lipofection, biolistic transfer, and electroporation. Suitable methods for transfecting host cells in vitro can be found in Sambrook et al., eds. (1989) Molecular Cloning: A Laboratory Manual (2nd ed., Cold Spring Harbor Laboratory Press, Cold Spring Harbor, N.Y.) and other laboratory manuals.

Screening Assays

The present split-hybrid system can be used for identifying compounds that disrupt protein-protein interactions. Compounds thus identified can be used to treat diseases involving abnormal protein-protein interactions.

The candidate compounds of the present invention can be obtained using any of the numerous approaches in combinatorial library methods known in the art. Such libraries include: nucleic acid libraries, peptide libraries, peptoid libraries (libraries of molecules having the functionalities of peptides, but with a novel, non-peptide backbone that is resistant to enzymatic degradation); spatially addressable parallel solid phase or solution phase libraries; synthetic libraries obtained by deconvolution or affinity chromatography selection; and the “one-bead onecompound” libraries. See, e.g., Zuckermann et al. (1994) J. Med. Chem. 37:2678-85 and Lam (1997) Anticancer Drug Des. 12:145.

Examples of methods for the synthesis of molecular libraries can be found in the art, for example, in: DeWitt et al. (1993) PNAS USA 90:6909; Erb et al. (1994) PNAS USA 91:11422; Zuckermann et al. (1994) J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner, U.S. Pat. No. 5,223,409), spores (Ladner, supra), plasmids (Cull et al. (1992) PNAS USA 89:1865-1869), or phages (Scott and Smith (1990) Science 249:386-390; Devlin (1990) Science 249:404-406; Cwirla et al. (1990) PNAS USA 87:6378-6382; Felici (1991) J. Mol. Biol. 222:301-310; and Ladner, supra).

To identify compounds that disrupt protein-protein interactions, a split-hybrid system described above is contacted with a candidate compound, and the expression level of the reporter gene is evaluated relative to that in the absence of the candidate compound. If the expression level of the reporter gene is greater in the presence of the candidate compound than that in the absence of the candidate compound, the candidate compound is identified as a potential drug for treating diseases involving abnormal protein-protein interactions.

The screening assay can be performed both in vitro and in vivo. Cell-based assays have notable advantages, e.g., the starting material (i.e., the cell) self-replicates, the biological context is closer to the normal physiological condition, and it provides insights into bioavailability (i.e., whether a compound can enter the cell to affect an intracellular target) and cytotoxicity (i.e., whether a compound compromises cell growth).

The specific examples below are to be construed as merely illustrative, and not limitative of the remainder of the disclosure in any way whatsoever. Without further elaboration, it is believed that one skilled in the art can, based on the description herein, utilize the present invention to its fullest extent. All publications recited herein are hereby incorporated by reference in their entirety.

Identification of Suppressors of Gene Expression

Gal4N-SMRT, Gal4N-Mad, and Gal4N-SHP (short heterodimer partner) were tested for their ability to suppress gene expression from a TK promoter containing 4 copies of Gal4 binding sites. Unexpectedly, Gal4N-SMRT and Gal4N-Mad were able to suppress the promoter activity, whereas Gal4N-SHP was not able to effectively block the promoter activity. These results suggest that SMRT and Mad can be used for establishing a split-hybrid system.

Retinoid Acid Split-Hybrid System

Two fusion proteins, VP16-RAR and Gal4N-IDII, were co-expressed in a cell containing a Gal-TK-Luc reporter gene construct. Unexpectedly, interactions between RAR and IDII led to a ˜120 fold induction of the reporter gene activity. In contrast, in the presence of 1 mM retinoid acid, interactions of IDII with RAR were blocked and induction of the reporter gene activity was reduced to only ˜7 fold. These results suggest that interactions between RAR and IDII are quite strong and can be disrupted by adding retinoid acid.

Next, two fusion proteins, NLS (nuclear localization signal)-HA (hemagglutinin)-Mad-RAR and Gal4N-IDII, were co-expressed in a cell containing a Gal-TK-Luc reporter gene construct. Unexpectedly, interactions between RAR and IDII led to a ˜7.5 fold repression of the reporter gene activity. The repression was completely reversed by adding retinoid acid (1 mM). Retinoid acid was specific in disrupting protein-protein interactions, as it did not significantly alter the reporter gene activity of Gal-TK-Luc by itself or in the presence of other co-transfection controls (i.e., Gal4N-Mad, Gal4N-IDII and NLS-HA-Mad-RAR).

Use of CMV and RSV Promoters

Other promoters such as CMV and RSV were tested to see whether they have higher basal activities that allow bigger windows for measuring the repressive effect. Five copies of the Gal4 binding site were placed to the 5′ end of the CMV or RSV promoter linked to the Luc reporter gene (Gal-CMV-Luc and Gal-RSV-Luc). Unexpectedly, the basal promoter activities of CMV and RSV were much higher than that of TK promoter.

Gal4N-SMRT and Gal4N-Mad conferred similar repression on Gal-TK-Luc. Interestingly, repression of Gal4N-SMRT on Gal-RSV-Luc was very robust (˜30 fold) compared to that on Gal-CMV-Luc. On the other hand, repressive effect of Gal4N-Mad on both promoters was not as efficient as on the TK promoter. These findings indicate that Gal-RSV-Luc with SMRT repressor domain is a better combination for establishing a split-hybrid system.

Next, two fusion proteins, SMRT-RAR and Gal4N-IDII, were co-expressed in a cell containing a Gal-RSV-Luc reporter gene construct. Unexpectedly, interactions of Gal4N-IDII with SMRT-RAR conferred 8.5-fold repression. This repression was reversed by retinoid acid treatment.

Another pair of interacting proteins, TRI and FKBP12, were also tested for use in a split-hybrid system. The interactions between these two proteins have been shown to be inhibited by small molecule FK506. The intracellular domain of TRI was fused to the Gal4 DNA binding domain (Gal4N-TRI). FKBP12 was linked to the repressor domain of SMRT (SMRT-FKBP12). Both plasmids were co-transfected with Gal4-RSV-Luc into CV-1 cells. Unexpectedly, interactions of Gal4N-TRI with SMRT-FKBP12 resulted in ˜12-fold repression of the reporter gene activity. Upon the FK506 treatment, protein interactions were blocked, leading to FK506 dosage-dependent activation of the promoter activity.

Use of SEAP and RED 1 as Reporter Genes

SEAP has been successfully used as a reporter gene in high throughput screen. To facilitate the high throughput screen using a split-hybrid system, a plasmid containing Gal-RSV-SEAP was constructed. Gal4N-SMRT and Gal-RSV-SEAP were co-transfected into CV-1 cells. Unexpectedly, Gal-SMRT conferred 14-fold repression of the reporter gene activity. Further, interactions between β-catenin and TCF repressed the SEAP reporter gene activity to the same extent as the Luc reporter gene activity.

Gal4N-SMRT effectively suppressed the expression of REDI, whereas Gal4N alone or the reporter protein alone conferred the fluorescent signal. Further, red fluorescence from RDE1 was detected in the presence of retinoid acid, RAR, and IDI1 or FK506, TRI, and FKBP12.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

What is claimed is:

1. A split-hybrid system comprising:
a nucleic acid containing a promoter, a transcriptional regulatory sequence operably linked to the promoter and containing a protein-binding domain, and a reporter gene operably linked to the promoter;
a first recombinant polypeptide including a DNA-binding domain that binds to the protein-binding domain and a bait polypeptide; and
a second recombinant polypeptide including a transcriptional repression domain and a prey polypeptide that interacts with the bait polypeptide;
wherein interaction between the bait and prey polypeptides represses the expression of the reporter gene, and disruption of the interaction between the bait and prey polypeptides activates the expression of the reporter gene.

2. The split-hybrid system of claim 1, wherein the promoter is a TK, CMV, or RSV promoter and the transcriptional repression domain is a Mad or SMRT domain.

3. The split-hybrid system of claim 1, wherein the protein-binding domain is a Gal4-binding site and the DNA-binding domain is Gal4N.

4. The split-hybrid system of claim 3, wherein the promoter is a TK, CMV, or RSV promoter and the transcriptional repression domain is a Mad or SMRT domain.

5. The split-hybrid system of claim 1, wherein the bait and prey polypeptides are prevented from interacting with each other by a compound that binds to the bait or prey polypeptide.

6. A cell comprising:
a first nucleic acid containing a promoter, a transcriptional regulatory sequence operably linked to the promoter and containing a protein-binding domain, and a reporter gene operably linked to the promoter;
a second nucleic acid encoding a first recombinant polypeptide including (1) a DNA-binding domain that binds to the protein-binding domain and (2) a bait polypeptide; and
a third nucleic acid encoding a second recombinant polypeptide including (1) a transcriptional repression domain and (2) a prey polypeptide that interacts with the bait polypeptide;
wherein interaction between the bait and prey polypeptides represses the expression of the reporter gene, and disruption of the interaction between the bait and prey polypeptides activates the expression of the reporter gene.

7. The cell of claim 6, wherein the promoter is a TK, CMV, or RSV promoter and the transcriptional repression domain is a Mad or SMRT domain.

8. The cell of claim 6, wherein the protein-binding domain is a Gal4-binding site and the DNA-binding domain is Gal4N.

9. The cell of claim 8, wherein the promoter is a TK, CMV, or RSV promoter and the transcriptional repression domain is a Mad or SMRT domain.

10. The cell of claim 6, wherein the bait and prey polypeptides are prevented from interacting with each other by a compound that binds to the bait or prey polypeptide.

11. The cell of claim 6, wherein the cell is a mammalian cell.

12. The cell of claim 11, wherein the promoter is a TK, CMV, or RSV promoter and the transcriptional repression domain is a Mad or SMRT domain.

13. The cell of claim 11, wherein the protein-binding domain is a Gal4-binding site and the DNA-binding domain is Gal4N.

14. The cell of claim 13, wherein the promoter is a TK, CMV, or RSV promoter and the transcriptional repression domain is a Mad or SMRT domain.

15. The cell of claim 11, wherein the bait and prey polypeptides are prevented from interacting with each other by a compound that binds to the bait or prey polypeptide.

16. A method of identifying a compound that disrupts interaction between a bait polypeptide and a prey polypeptide, the method comprising providing a split-hybrid system of claim 1 and contacting the system with a compound, wherein the expression level of the reporter gene, if higher in the presence of the compound than in the absence of the compound, indicates that the compound disrupts the interaction between the bait and prey polypeptides.

17. A method of identifying a compound that disrupts interaction between a bait polypeptide and a prey polypeptide, the method comprising providing a cell of claim 6 and contacting the cell with a compound, wherein the expression level of the reporter gene, if higher in the presence of the compound than in the absence of the compound, indicates that the compound disrupts the interaction between the bait and prey polypeptides.

18. The method of claim 17, wherein the cell is a mammalian cell.
